(19) United States
(12) Patent Application Publication
(10) Pub. No.: US 2024/0389839 A1
(43) Pub. Date: Nov. 28, 2024

(54) AN ENDOSCOPE

(71) Applicant: AMBU A/S, Ballerup (DK)

(72) Inventors: Søren PEDERSEN, Bagsværd (DK); Victor SILBERMANN, Them (DK)

(73) Assignee: AMBU A/S, Ballerup (DK)

(21) Appl. No.: 18/695,339

(22) PCT Filed: Sep. 22, 2022
(86) PCT No.: PCT/EP2022/076407

§ 371 Date: Mar. 25, 2024

(30) Foreign Application Priority Data
Sep. 28, 2021 (DK) PA 2021 70472

(51) Int. Cl. A61B 1/005; A61B 1/00

(52) U.S. Cl. CPC A61B 1/0057; A61B 1/00042; A61B 1/00045; A61B 1/00128; A61B 1/0052

(57) ABSTRACT

An endoscope including a proximal handle and an insertion cord (6). The handle includes an operating member (81) for the bending section. A first pull-wire and a second pull-wire are connected to the bending section. The first pull-wire partially is surrounded by a first guide tube so as to present a first proximal pull-wire section (191a) extending from a proximal end (193a) said first guide tube and the second pull-wire is partially surrounded by a second guide tube so as to present a second proximal pull-wire section (192a) extending from a proximal end of said second guide tube (194a). The first pull-wire section (191a) is in engagement with a wire guide on the operating member (81) and connected to the operating member, and the first pull-wire section (191a) is joined with the second pull-wire section (192a) at a location between the operating member (81) and the proximal end (194a) of the second guide tube.

### CROSS-REFERENCE TO RELATED APPLICATIONS

This application is a National Stage entry under 35 U.S.C. § 371 of International Application No. PCT/EP2022/076407, filed Sep. 22, 2022, which claims priority from and the benefit of Danish Patent Application No. PA 2021 70472, filed Sep. 28, 2021; the disclosures of said applications are incorporated by reference herein in their entirety.

### TECHNICAL FIELD

The present disclosure relates to endoscopes in general and more specifically to means for maintaining a tensioned pull wire in an endoscope.

### BACKGROUND

Endoscopes are well known devices for visually inspecting inaccessible places such as human body cavities. Typically, the endoscope comprises an elongated insertion cord with a handle at the proximal end as seen from the operator and visual inspections means, such as a built-in camera, at the distal end of the elongated insertion cord. This convention of distal and proximal, proximal being the end closest to the operator and distal being the end remote from the operator, as used above for the endoscope in general will, where applicable, be adhered to for all parts throughout this description. Electrical wiring for the camera and other electronics such as LED lighting run along the inside of the elongated insertion tube from the handle to the tip at the distal end. Instead of using cameras, endoscopes may also be fibre-optic, in which case the optical fibres run along in-side of the elongated insertion tube. Also, a working channel may run along the inside of the insertion cord from the handle to the tip, e.g. allowing liquid to be removed from the body cavity or allowing the insertion of surgical instruments or the like into the body cavity.

Furthermore, in order to be able to manoeuvre the endoscope inside the body cavity, the distal end of the endoscope may comprise bending section, i.e. a section with increased flexibility, such an articulated tip part allowing the operator to bend this section. Typically, this is done by tensioning or slacking pull wires in a guide tube also running along the inside of the elongated insertion cord from the articulated tip part to a control mechanism with a control knob in the handle in an arrangement commonly known as a Bowden cable.

The pull wire running along the inside of the guide tube of a Bowden cable normally extends with a predetermined length over either end, allowing an operating member to be attached to a free end, in the following referred to as the proximal end, and an operated member to be attached to the other free end, in the following referred to as the distal end. When the ends of the guide tube are held stationary, movement of the proximal end of the pull wire with respect to the guide tube is transmitted to the distal end as a corresponding movement of the distal end of the pull wire with respect to the guide tube, so as to effect a movement of the operated member.

When assembling the endoscope, care needs to be taken that the pull wires have the correct length from the operating member to the operated member and the correct tension so that there is no slack. That is to say, the length of the pull wires must ensure that a neutral middle position of the operating member corresponds to a straight unbent state of the bending section.

In this respect, US2020/0229684 discloses a disposable endoscope in which two pull wires are each secured at one end at the operated member. During assembly, each of the two pull wires is threaded through a suitable hole or eyelet in the operating member and the free end pulled back along the pull wire itself under suitable tension. The free end is then crimped onto the pull wire itself to form a loop, ensuring the correct length and tension at assembly as well as during subsequent storage and use of the disposable endoscope, the latter being important as single use endoscopes must be ready for use when needed and not need prior adjustment or calibration.

Furthermore, US2019/0231179 discloses an endoscope where two pull wires are secured to the operating member using pins once the pull wires have been put under the desired tension. Though this solution has proved successful there is still room for improvement when it comes to simplifying the assembly of the disposable endoscope. Simplicity is important to inter alia keep the assembly costs down and, in turn, the price of the endoscope down.

### SUMMARY

It is the object of the present disclosure to provide an endoscope with low cost parts that render themselves for easy assembly procedures during manufacture.

According to a first aspect of the disclosure, this object is achieved by an endoscope comprising a proximal handle and an insertion cord, where the insertion cord comprises a bending section at the distal end of the insertion cord and where the handle comprises an operating member for said bending section, where a first pull-wire and a second pull-wire are connected to the bending section, where the first pull-wire partially is surrounded by a first guide tube so as to present a first proximal pull-wire section extending from a proximal end said first guide tube and the second pull-wire is partially surrounded by a second guide tube so as to present a second proximal pull-wire section extending from a proximal end of said second guide tube, where the first pull-wire section is in engagement with a wire guide on the operating member and connected to the operating member, and where the first pull-wire section is joined with the second pull-wire section at a location between the operating member and the proximal end of the second guide tube.

Joining the two pull-wires at this location achieves several advantages. First of all, this is an easy location to access. Furthermore, it is easy to align the two proximal pull-wire sections with each other and put them under tension. Also, it allows for one joining location only that can be easily made while the pull-wires are both concurrently under correct tension. Moreover, it allows for final adjustment of the operating member to a neutral or middle position corresponding to a straight, unbent, state of the bending section.

According to a second aspect of the disclosure, the object is also achieved by a method in assembling an endoscope, said method comprising providing a first handle housing part, mounting an operating member in said first handle housing part, providing a bending section, connecting a first pull-wire and a second pull-wire to the bending section, placing the first pull-wire in and partially surrounded by a first guide tube so as to present a first proximal pull-wire section extending from a proximal end said first guide tube, placing the second pull-wire in and partially surrounded by a second guide tube so as to present a second proximal pull-wire section extending from a proximal end of said first guide tube, placing the first pull-wire section in engagement with at least one wire guide provided on said operating member, tensioning the first pull-wire to a predetermined tension, tensioning the second pull-wire to a predetermined tension, while under tension joining the first proximal pull-wire section with the second proximal pull-wire section, adjusting the position of the operating member with respect to the first pull-wire, and securing the position of the first pull-wire with respect to the operating member.

According to a third aspect of the disclosure, this object is also achieved by a system comprising a display device and an endoscope according to the first aspect of the disclosure connectable to the display device.

According to an embodiment of the first aspect of the disclosure, endoscope according to any one of the preceding claims wherein the operating member comprises a central axle. The central axle makes it easy to provide a rotary operating member by simply click the operating member into place in suitable trunnions or the like provided in the handle housing.

According to an embodiment of the first aspect of the disclosure, the operating member comprises a first hook and a second hook arranged at a distance from said central axle and an defining a minor and a major angular spacing with respect to each other, said pull-wires being attached to the operating member at an angle within said minor angular spacing. These hooks provide a labyrinth with sufficient friction to facilitate correct positioning and to keep the joined pull-wires in that position when released for final securing.

According to an embodiment of the first aspect of the disclosure, the first pull-wire section is joined with the second pull-wire section by a crimp member. Using a single crimp member to join the two pull-wires in an easy to access location greatly facilitates the assembly process for the endoscope.

According to an embodiment of the first aspect of the invention, the first pull-wire is attached to the operating member by means of a rivet. Using a rivet is advantageous in that the rivet stem and rivet head may first be used as a wire guide when locating the first proximal pull-wire section on the operating member and the rivet then be used to secure the first pull-wire permanently to the operating member in the correct location ensuring a neutral position of the operating member when the bending section is in the straight unbent state.

According to an embodiment of the first aspect of the disclosure, the rivet is a blind rivet. A blind rivet is easy to place in a suitable bore in the operating member where it will serve as a wire guide for the first pull-wire and easy to secure with commonplace tools in the location where it will secure the first pull-wire, and along with it the second pull-wire, in the correct position with respect to the operating member.

According to an embodiment of the first aspect of the disclosure, the wire guide comprises at least one aperture in said operating member through which the first pull-wire section passes. This aids in ensuring alignment with the operating member, typically comprising a roller, and preventing lateral slippage.

According to an embodiment of the first aspect of the disclosure, the wire guide comprises a plurality of apertures through which the first pull-wire passes, said apertures being located at different angles within said major angular spacing. These prevent the lateral slipping and the correct alignment of the first pull-wire in use.

According to an embodiment of the disclosure, the first pull-wire and the second pull-wire are constituted by one and the same physical wire. Using only one single physical wire facilitates the number of parts to be handled during assembly, reduces the number of connections to be made to the bending section, and the threading of wired therethrough, thus in general facilitates the assembly process.

According to an embodiment of the second aspect of the invention, the connecting of the first proximal pull-wire section to the second proximal pull-wire section is achieved using a crimping member. Using a single crimp member suffices to connect the two pull-wires in an easy to access location and greatly facilitates the assembly process for the endoscope. Because a loop is easily formed around the operating member, the loop, in turn, facilitates the adjusting of the operating member with respect to the first pull-wire. Because of the connection, the operating member will automatically also be adjusted with respect to the second pull wire.

According to an embodiment of the second aspect of the disclosure, the securing of the position of the first pull-wire with respect to the operating member is performed by riveting. This is an easy way involving only common tools to secure the first pull-wire with respect to the operating member, in particular, when as preferred the rivet is a blind rivet. Because the first and second pull-wires are mutually connected to each other at their proximal end sections, placing a single rivet suffices to secure the position of the operating member with respect to both.

According to an embodiment of the second aspect of the invention, the placing of the first pull-wire section in engagement with the at least one wire guide comprises threading it through an aperture provided on said operating member. This laterally secures the first pull-wire with respect to the operating member, ensures alignment and prevents lateral slippage.

### BRIEF DESCRIPTION OF THE FIGURES

The disclosure will now be made in greater detail based on non-limiting exemplary embodiments and with reference to the drawings on which:

FIG. 1 shows a system comprising a display unit and an endoscope according to the disclosure,

FIG. 2 shows an exploded view of the endoscope according to the disclosure,

FIG. 2A shows an exploded view of the handle and connection cable of the endoscope of FIG. 2

FIG. 2B shows an exploded view of the insertion cord of the endoscope of FIG. 2,

FIG. 3 shows the handle of the endoscope of FIG. 2 with one handle housing part removed for visibility,

FIG. 4 shows the operating member, the pull-wires in guide tubes and an anchoring block for the guide tubes seen from one side,

FIG. 5 shows the operating member, the pull-wires in guide tubes and an anchoring block for the guide tubes as seen from the opposite side,

FIG. 6 shows the endoscope with the pull-wires to the bending section with parts removed for visibility,

FIG. 7 shows an isometric view of one side the operating member from one side,

FIG. 8 shows an isometric view of the opposite side of the operating member of FIG. 7,

FIG. 9 shows an isometric view of the operating member of FIG. 7 from the same side as FIG. 8 but from a different angle,

FIG. 10 shows a first radial view of the operating member of FIG. 7,

FIG. 11 shows a second radial view of the operating member of FIG. 7 form a different angle, and

FIG. 12 shows the angular layout of the wire guide.

### DETAILED DESCRIPTION

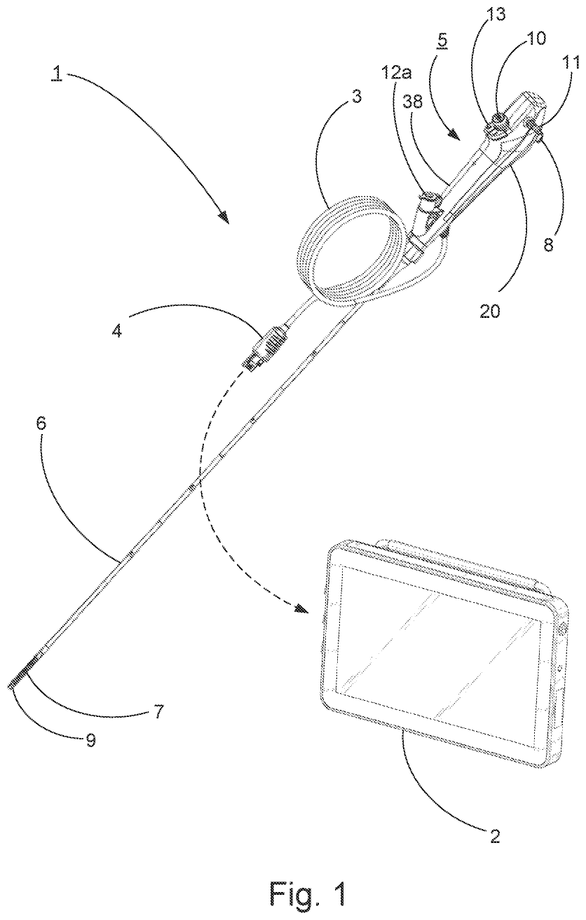

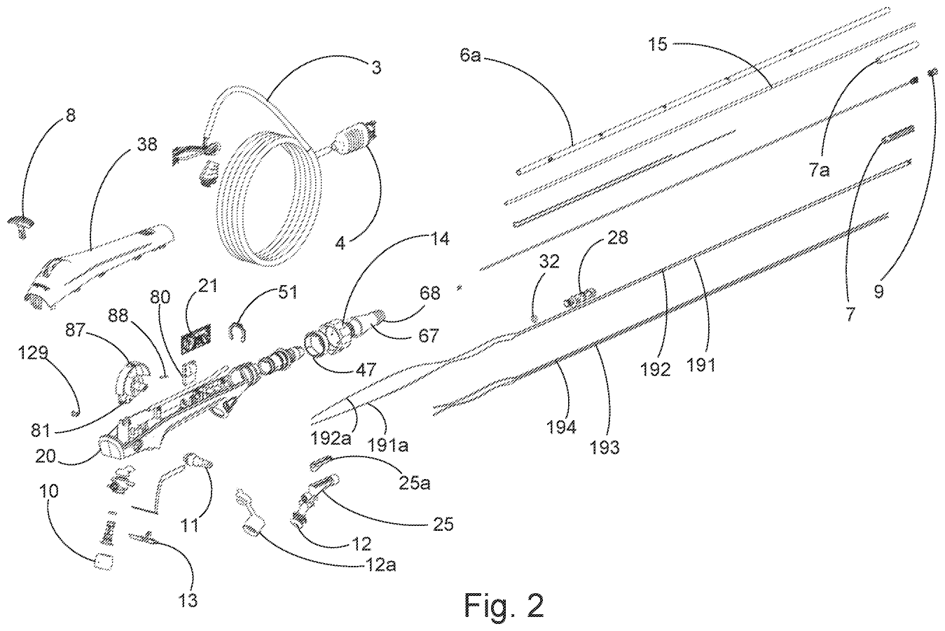

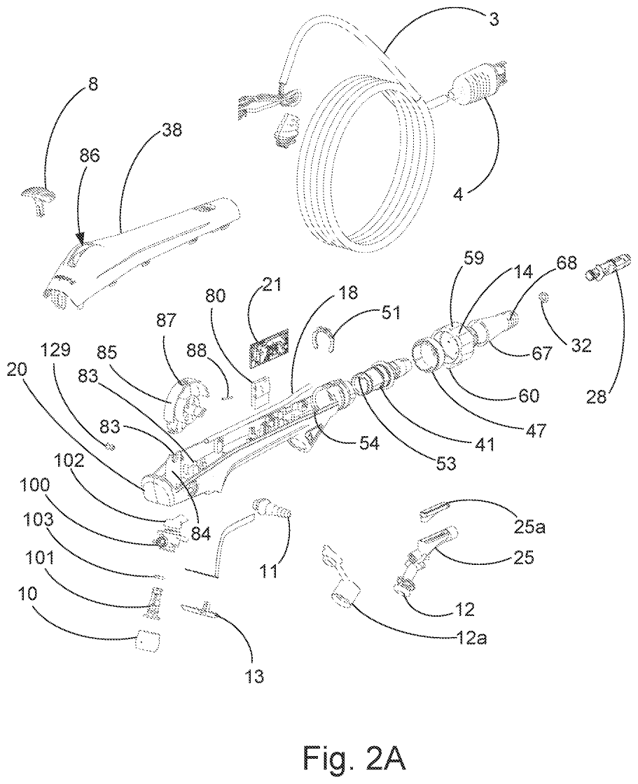

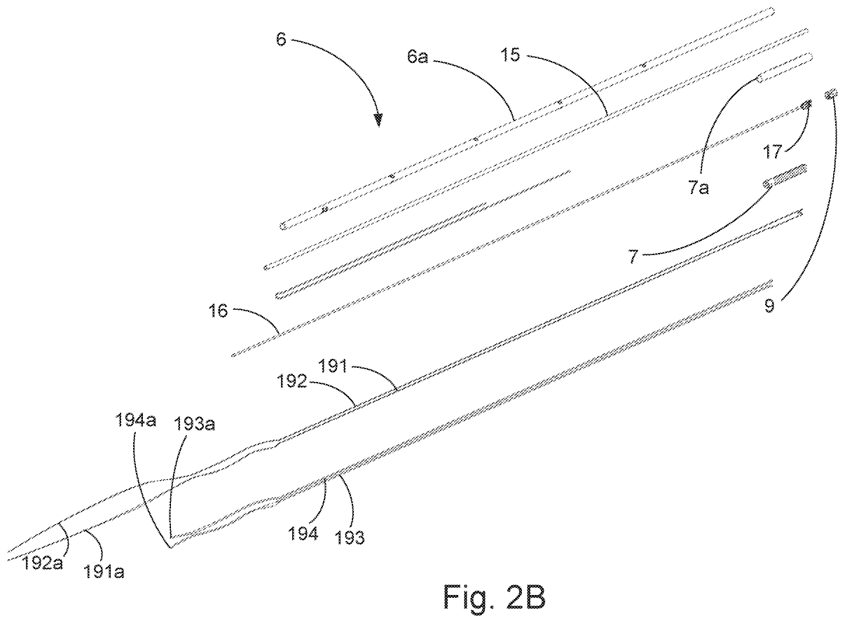

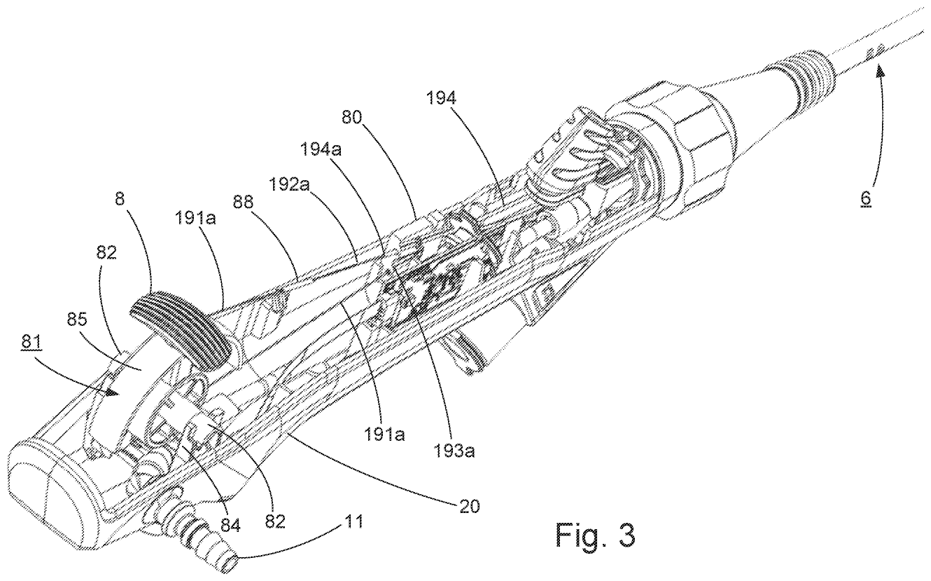

Turning first to FIG. 1, a system comprising an endoscope 1 and a display unit 2 is shown. The endoscope 1 may be connected to the monitor 2 by means of a cable 3 with a suitable connector 4. The endoscope 1 is preferably disposable i.e. intended to be thrown away after use on one single patient, whereas the display unit 2 may be used multiple times with different reusable endoscopes. The endoscope 1 is an insertion endoscope comprising a handle 5 at the proximal end, and an insertion cord 6 extending from the handle 5 towards the distal end of the endoscope 1. The handle preferably comprises two major constituent parts, i.e. main handle housing part 20 and a lid part 38. At the distal end of the insertion cord 6 a bending section which may be controlled by an operating knob 8 is provided. As can be seen from FIGS. 2 and 2B the bending section comprises a bending section body 7 beneath a flexible cover 7a. In turn at the distal end of the bending section 7, a tip housing 9 with the image capture device, lenses, illumination, the distal port of a working and/or suction cannel etc. of the endoscope 1 is provided. At the distal end the working channel will normally double as both working channels for tools and suction channel. The handle 5 normally also comprises a suction activation button 10 for activating the suction though the suction channel to an external vacuum source (not shown) via a suction connector 11. The handle may also have a tool insertion port 12 (cf. FIG. 2) with a cap 12a for the insertion of an external tool through the handle and the working channel so as to emerge from the distal working channel port at the tip housing 9. Also, the handle 5 may be provided with buttons for activation electrical switches 13 controlling function of the image capture, such as taking of still images.

To control the bending section 7 the endoscope 1 comprises an internal arrangement of pull-wires comprising a first pull-wire 191 and a second pull-wire 192 as can e.g. be seen from the exploded view of FIG. 2. For practical purposes the first-pull wire 191 and second-pull wire 192 may physically be one single wire body, which as soon as it has been threaded though and are suitably connected to the distal end of the bending section 7 cannot, from a pulling perspective, which is the purpose in this disclosure, be distinguished from two individually fixed pull-wires 191, 192. That is to say, even though there is conceptually and functionally a first pull-wire and a second pull-wire they are as a constituent part of the endoscope constituted by one and the same physical wire.

The pull-wire arrangement is devised as Bowden cables and accordingly the first pull-wire 191 is partially surrounded by a first guide tube 193. That is to say the first guide tube 193 has a shorter length than the first pull-wire 191 so that a first proximal pull-wire section 191a extends from the proximal end 193a of said first guide tube 193. Likewise, the second pull-wire 192 is partially surrounded by a second guide tube 194, also having a shorter length than the second pull-wire 192 so that a second proximal pull-wire section 192a extends from the proximal end 194a of said second guide tube 194.

At or close to their proximal ends 193a, 194a, the first and second guide tubes 193, 194 are secured in an anchoring block 80 preferably accommodated in a receptacle provided in the inside wall of the main handle housing part. The anchoring block 80 is preferably made of a transparent material with suitable passages for the guide tubes 193, 194 and inlets for an adhesive used for securing the guide tubes 193, 194. As can best be seen from FIG. 5, the passages for the guide tubes 193, 194 are preferably at an angle to each other so that the first proximal pull-wire section 191a extends in a first predetermined direction from the proximal end 193a of said first guide tube 193 towards an operating member 81, and so that the second proximal pull-wire section 192a extends in a second predetermined direction from the proximal end 194a of said first guide tube 194 towards the operating member 80. This arrangement of the Bowden cables and the anchoring block 80 essentially corresponds to the arrangement in the aforementioned prior art of US2020/0229684, which is incorporated herein by reference.

Also shown in FIGS. 2 and 2A are the following parts: a connection member 28, a sealing member 32, a tension relief member 67 with circumferential elastic ribs 68, a gripping ring 14 with teeth 59, 60, a marker ring 47, a bushing 41 with a stop member 53, a C-shaped clip 51, handle housing uprights 54, a circuit board 21, and a Y-junction body 25 with a Y-junction lid 25a.

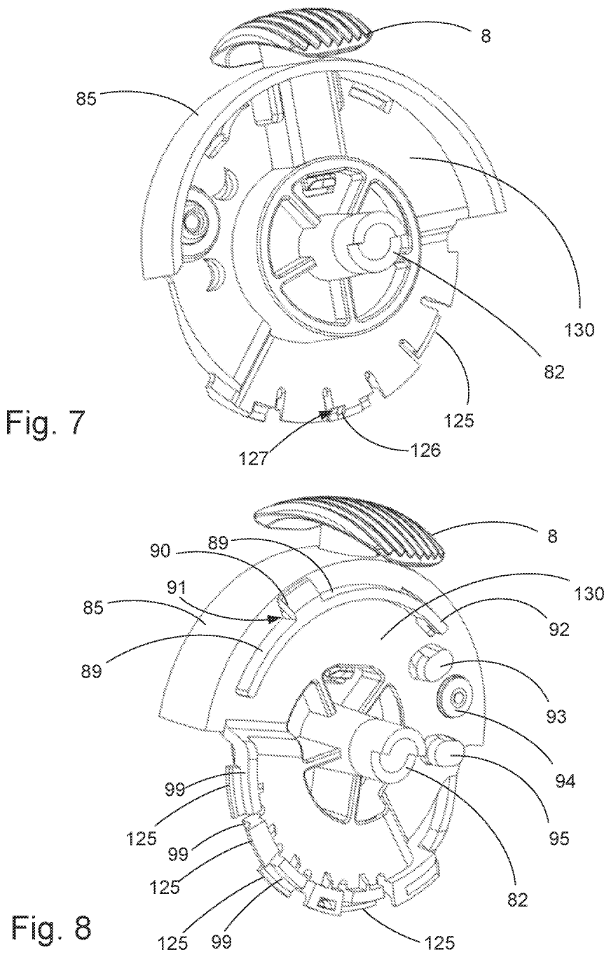

Turning now to FIGS. 7-11, the operating member 81 is shown in greater detail from various angles. The purpose of the operating member 81 is to transfer force applied by a finger of an operator to an operating means 8, such as a knob or lever, extending from the operating member 81 to the exterior of the handle 5 to the pull-wires 191, 192 in order to pull the bending section 7 to one side or the other in response to the force applied by the operator.

The operating member 81 is therefore provided with a central axle 82 about which the operating member 81 may rotate with respect to the handle housing, once the ends of the central axle 82 are clicked elastically into place in suitable bearings provided as cut-outs 83 in uprights 84 formed along and as part of the inner wall of the main handle housing part 20. The axle is surrounded by a wall 130 extending in the radial direction. The depicted operating member 81 may somewhat imprecisely be described as disc, roller or pulley shaped but other shapes are possible without deviating from the present disclosure.

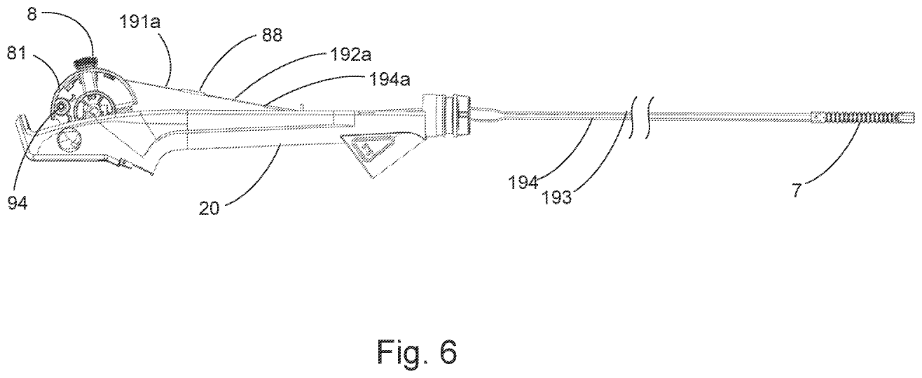

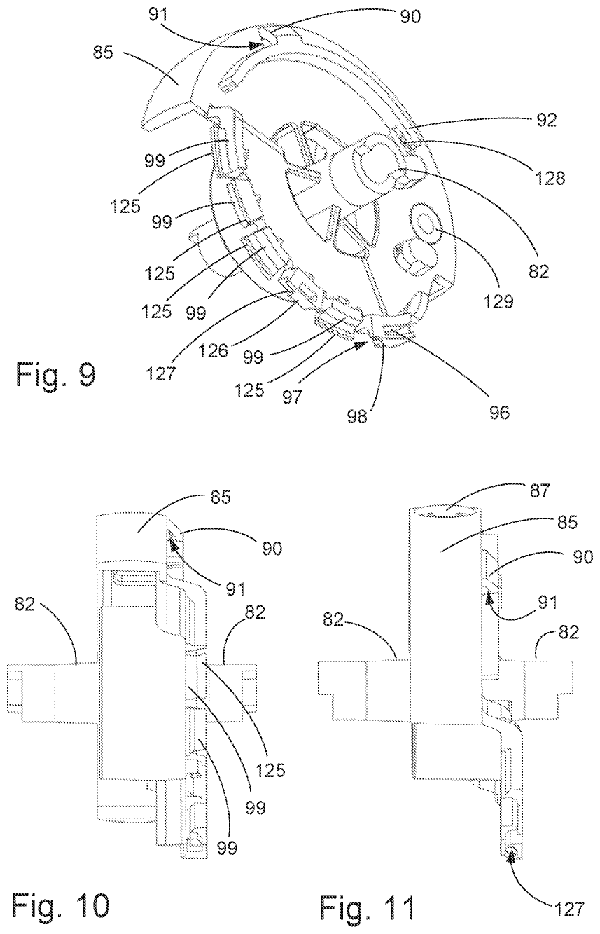

The operating member 81 comprises a shield surface 85 formed as approximately a half cylinder and serving to protect the interior against ingress of liquids and pollutants through the slit-shaped opening 86 provided in the lid part 38 of the handle 5. The operating means 8 extends through this opening 86 and is received into a receptacle 87 provided in the shield surface 85. The operating member 81 is so designed that it may be moulded as one single item in a mould where the mould parts may be separated along the axis of the axle 82, the receptable 87 being the only feature necessitating an additional form part, such as a radially retractable core. The operating member 81 may preferably be moulded with an insert 129, such as a cylindrical metal bushing of brass or the like to provide a reinforced hole in the wall 130, cf. FIG. 9, serving to provide hold for a fastening member 94 to be described below. Preferably, the hole or the insert 129 is provided in the upper part of the operating member 80, so as to extend over the rim of the main handle housing part 20, as scan be seen in FIG. 6, for easy access to position and attachment of a fastening member 94 in the hole, when the operating member 80 is placed in the bearings 83. The easy access allows for the use of a riveting tool.

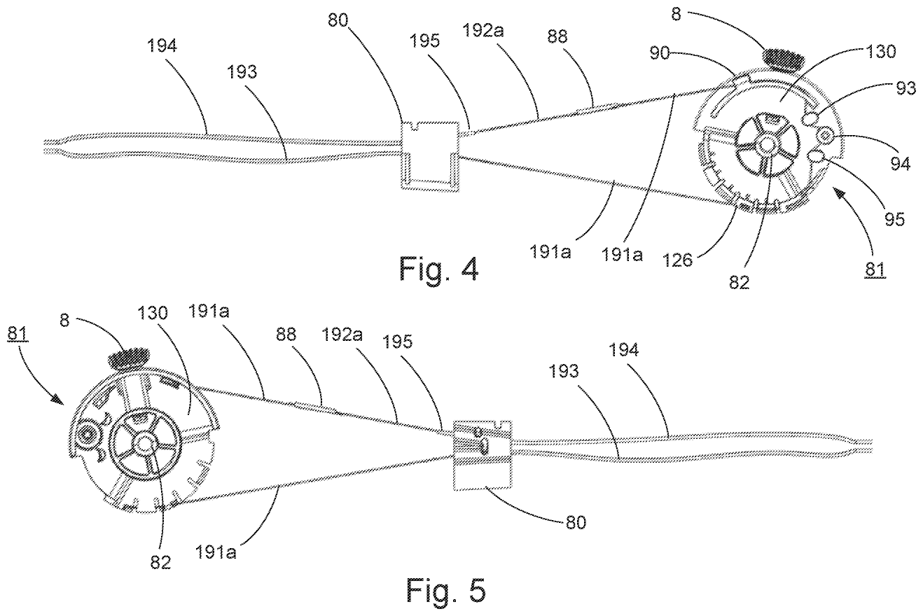

As can be seen from e.g. FIGS. 4 and 5, the first proximal pull-wire section 191a is joined with the second proximal pull-wire section 192a so that a loop is formed around axle 82. The loop is formed at a distance from the axle 82 for leverage. More specifically, the proximal pull-wire section of the first pull-wire 191 is held in a wire guide at various radial distances from the axle 82. As will be explained below, the loop is formed by joining the first proximal pull-wire section 191a with the second proximal pull-wire section 192a by a suitable connection member, preferably a crimp 88 crimped onto the overlapping parts of the pull-wires 191, 192, using pliers or the like. This joint is made at an easily accessible place at a location between the operating member 80 and the proximal end 194a of the second guide tube 194.

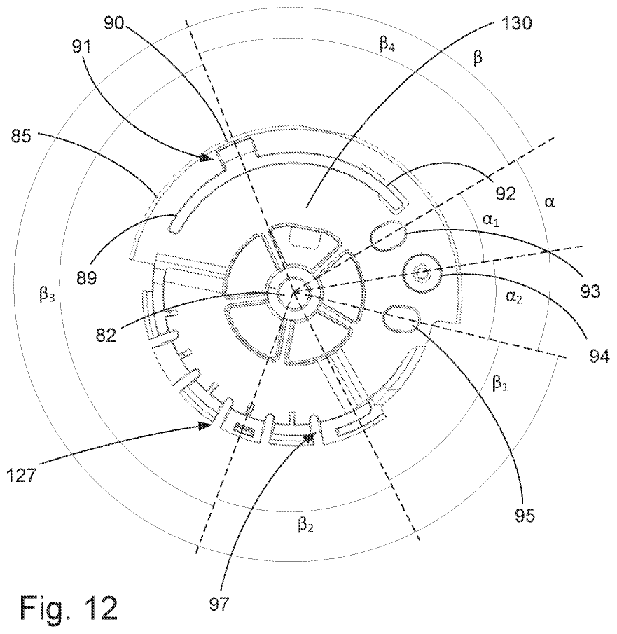

The wire guide may comprise several different features or guide parts provided on the operating member 81. Starting clockwise the wire guide comprises a ledge 89 adapted to keep the first pull-wire 191 at a predetermined distance from the axle 82. A portal 90 with an aperture 91 through which the first pull-wire 191 passes is provided to secure the first pull-wire in the lateral direction and inter alia prevent it from slipping off the ledge 89. The ledge 89 continues on the other side of the portal 90, where it has a partial taper to provide a free arm 92 with a barb 128. This taper guides the first pull-wire 191 towards a radially inwardly pointing first hook 93 provided on the wall 130, and under which hook 93 the first pull-wire 191 passes. As can be seen in FIG. 12, the first hook 93 is arranged at a distance from the centre axle 82, and with an angular spacing β₄ from the portal 90. From the first hook 93 the first pull-wire 191 takes an outward curve around the stem of a fastening member, such as a rivet 94, arranged in a suitable hole in the wall 130 in the axial direction of the axle 82 of the operating member 80. As can be seen from FIG. 12, the first hook 93 and a second hook 95 are arranged at a radial distance from the centre axle 82, and have an angular spacing α (or β depending on how you look at it), where α<β, and the fastening member is arranged in the angular spacing α, i.e. in the minor of the α and β angular spacings. The angular location of the fastening member is preferably so that a is divided equally into a and da. The first pull-wire passes under the head of the fastening member, which so secures the first pull-wire 191 in the lateral direction, i.e. in the direction of the axis of the axle 82. The first pull-wire then passes under the second hook 95 provided in the wall 130, essentially identical to the first hook 93. This path of the first guide wire 191 essentially lies in one and the same plane along the wall 130 and perpendicular to the axis of the axle 82.

As will be noticed from e.g. FIGS. 7 and 8, the upper part of the operating member (as seen in those Figures) is axially off-set with respect to the lower part. This is primarily to provide space for a suction valve arrangement 100, 101, 102 and the associated suction tube 18.

Accordingly, from the second hook 95 the path of the first pull-wire 191 moves out of the previous plane onto a ramp surface 96 and through an aperture 97 in a second portal securing it laterally in a new plane. The aperture 97 is arranged in the major of the α and β angular spacings, approximately at the angle 31. In the new plane the wire guide comprises a number of teeth with ledges 99 and flanges 125. Some of the teeth may also comprise portals 127 with apertures 126 through which the first-pull wire 191 passes. From the last portal 127 clockwise, i.e. approximately at the angle β₁+β₂ from the second hook 95, onward the first pull-wire 191 may only engage and be guided by the ledges 99 and flanges 125 in some of the angular positions of the operating member 80 when it is turned during use.

As for the assembly, it is preferred that the first and second pull-wires 191 and 192 are provided as a prefabricated insertion cord subassembly. That is to say an insertion cord subassembly comprising a bending section 7 through which the first and second pull wires 191 have been threaded and secured, e.g. to the most distal end or segment of bending section 7 and secured, be it as one single wire body or two separate ones. The pull-wires 191, 192 are inserted into the respective guide tubes 193, 194 so that the first proximal pull-wire section 191a of the first pull-wire 191 protrudes from the proximal end of the first guide tube 193 and the second proximal pull-wire section 192a of the second pull-wire 192 protrudes from the proximal end of the second guide tube 194, thus forming Bowden cables. These Bowden cables are inserted into a main tube 6a that surrounds them over some of the length but with both guide tubes 193,194 pull-wires 191, 192 extending from the proximal end. In the main tube a working channel tube 15 and electrical signal and supply wires 16 leading to an imaging device 17 is also accommodated. The working channel tube 17 and the signal and supply wires 16 also extend from the proximal end of the assembled insertion cord subassembly 6. At the distal end the insertion cord a tip housing 9 is located. The tip housing is connected with the bending section 7, the working channel tube 15, and the main tube. The tip housing 9 also accommodates the imaging device 17. An outer sleeve 7a surrounds the distal end and in particular the bending section, which is normally an open articulated structure, against ingress of fluids and pollutions.

This finished insertion cord subassembly is then connected to the distal end of the main handle housing 20. The first and second guide tubes 193, 194 are then placed in the anchoring block and secured in position, e.g. using an UV curing adhesive and ultraviolet irradiation.

The first proximal pull-wire section 191a is then placed in the wire guide, e.g. by threading it through the apertures 91, 97, 127 and wrapping it around the first and second hook 93, 94 as well as the stem of the fastening member, such as a blind rivet, as described above.

The operating member is then clicked into place in the bearings 83 so that the first proximal pull-wire section extends towards and past the second proximal pull-wire section, in a close, preferably parallel relationship allowing a crimp 88 or other connection member to be placed around both.

While holding the bending section 7 in a straight configuration, by clamping or the like, the free ends of both the first proximal pull-wire section 191a and the second pull-wire section 192b may now be put under the desired tension, ensuring that there is no slack in the Bowden cables. This tensioning can simply be a wire over a pulley with a predetermined weight at one end and a clip at the other for temporary attachment to the respective pull-wire. Securing the first proximal pull-wire section 191a to the second proximal pull-wire section 192, using e.g. pliers to crimp the crimp, or any other suitable means to join the first proximal pull-wire section 191a to the second proximal pull-wire section 192 will now ensure correct tensioning of the Bowden cables.

The first proximal pull-wire section is however still able to slip with respect to the operating member in the wire guide. This is desired, as it allows the angular positioning of the operating member 80 so that the receptacle 87 is aligned with the centre of the slit 86 along the length thereof once the lid part 38 is mounted, thus ensuring the operating means 8 will be in a neutral middle position in the slit 86. Since the bending section 7 is still held straight during this alignment and the tension on any of the pull-wires 191, 192 do not change because they are secured to each other, this neutral position will in use correspond with the unbent or neutral state of the bending section 7.

In this neutral position a fastening member, such as a rivet 94, may then be secured to attach the first pull wire to the operating member. This permanently secures the first pull-wire 191 to the operating member 80 in turn securing that the straight, unbent state of the bending section corresponds to the neutral position of the operating means 8. Using a blind rivet 94 placed in a corresponding hole in the operating member 80, preferably a hole lined with a metal bushing, is an easy and efficient way of achieving this without any need for specialized tools purpose-built for the assembly of the endoscope 1.

With the pull-wire arrangement (and other parts) so in place, the lid part may 38 be placed over the main handle housing part 20 to close the handle 5, and the operating means 8 inserted into the receptacle 87 through the slit 86 to complete the assembly.

The previous description has been given based on preferred exemplary embodiments, but it will be clear for the skilled person that many deviations and variants are possible without deviation from the scope of the claim. In particular the wire guide may comprise entirely different features as long as they allow the temporary slippage of the first pull-wire for adjustment of the operating member until finally secured. Likewise, the securing may be made with essentially any kind of fastener that may constitute part of the wire guide and not only using a rivet. Also, the joining of the first pull-wire to the second pull-wire may be made by other means than crimping.

We claim:

1-13. (canceled)

14. An endoscope comprising:
a handle including an operating member, the operating member comprising a wall, the wall including an arcuate ledge, a first hook, and a second hook, the first hook and the second hook being separated by a minor angular spacing having a minor angle and a major angular spacing having a major angle, the minor angle and the major angle being conjugate angles;
a first pull-wire comprising a proximal section and a distal end, the proximal section extending along the arcuate ledge, partially around the first hook, and partially around the second hook, and the proximal section being affixed to the wall within the minor angular spacing;
a second pull-wire comprising a proximal section and a distal end, the proximal section of the first pull-wire being affixed to the proximal section of the second pull-wire; and
an insertion cord including a bending section, the bending section being connected to the distal end of the first pull-wire and to the distal end of the second pull-wire.

15. The endoscope of claim 14, wherein the arcuate ledge, the first hook, and the second hook protrude from one side of the wall.

16. The endoscope of claim 14, wherein the arcuate ledge, the first hook, and the second hook protrude from one side of the wall, wherein the operating member further comprises a portal extending radially outwardly from the arcuate ledge, wherein the portal comprises an aperture, wherein the arcuate ledge comprises a free arm with a barb, and wherein the proximal section of the first pull-wire passes through the aperture and between the wall and the free arm.

17. The endoscope of claim 14, wherein the wall comprises a hole,
wherein the endoscope further comprises a fastening member including a stem and a head, the stem received by the hole in a first position and in a second position of the fastening member,
wherein the arcuate ledge, the first hook, the second hook and the head protrude from one side of the wall,
wherein the proximal section of the first pull-wire extends slidingly, while the fastening member is in the first position, along the arcuate ledge, partially around the first hook, partially around the stem, and partially around the second hook, and
wherein, when the fastening member is in the second position, the fastening member affixes the proximal section of the first pull-wire to the operating member and prevents the proximal section of the first pull-wire from sliding along the arcuate ledge.

18. The endoscope of claim 17, wherein the fastening member comprises a rivet.

19. The endoscope of claim 17, wherein the fastening member comprises a blind rivet.

20. The endoscope of claim 14, wherein the operating member further comprises a portal extending radially outwardly from the arcuate ledge.

21. The endoscope of claim 20, wherein the portal comprises an aperture, and wherein the proximal section of the first pull-wire passes through the aperture.

22. The endoscope of claim 14, wherein the operating member comprises a central axle, wherein the wall comprises a first section and a second section, the first section extending along a first plane perpendicular to the central axis, and the second section extending along a second plane offset from the first plane, and wherein the arcuate ledge and the first hook protrude from one side of the first section of the wall.

23. The endoscope of claim 22, wherein the second section of the operating member comprises teeth including ledges and flanges, the ledges and flanges guiding the proximal section of the first pull-wire.

24. The endoscope of claim 23, wherein at least some of the teeth comprise portals with apertures, the proximal section of the first pull-wire passing through said apertures of said portals of said teeth.

25. The endoscope of claim 14, wherein the operating member further comprises a wire guide including a plurality of apertures, and wherein the proximal section of the first pull-wire passes through the plurality of apertures, said apertures being located at different angles, relative to the central axis, within said major angular spacing.

26. The endoscope of claim 14, wherein the first pull-wire and the second pull-wire are constituted by one and the same physical wire.

27. The endoscope of claim 14, wherein the first pull-wire is partially surrounded by a first guide tube, the proximal section of the first pull-wire extending proximally from the first guide tube, wherein the second pull-wire is partially surrounded by a second guide tube, the proximal section of the second pull-wire extending proximally from the second guide tube, and wherein the first guide tube and the second guide tube are affixed to an anchoring block.

28. A system comprising:
a display device; and
the endoscope of claim 14, the endoscope being connectable to said display device.

29. An endoscope comprising:
a handle including an operating member, the operating member comprising a wall, the wall including an arcuate ledge, a first hook, a second hook, and a hole, the first hook and the second hook being separated by a minor angular spacing having a minor angle and a major angular spacing having a major angle, the minor angle and the major angle being conjugate angles, the hole positioned within the minor angular spacing;
a fastening member including a stem and a head, the stem received by the hole in a first position and in a second position of the fastening member;
a first pull-wire comprising a proximal section and a distal end;
a second pull-wire comprising a proximal section and a distal end; and
an insertion cord including a bending section, the bending section being connected to the distal end of the first pull-wire and to the distal end of the second pull-wire,
wherein the proximal section of the first pull-wire extends slidingly, while the fastening member is in the first position, along the arcuate ledge, partially around the first hook, partially around the stem, and partially around the second hook,
wherein, when the fastening member is in the second position, the fastening member affixes the proximal section of the first pull-wire to the operating member and prevents the proximal section of the first pull-wire from sliding along the arcuate ledge, and
wherein the proximal section of the first pull-wire is affixed to the proximal section of the second pull-wire.

30. The endoscope of claim 29, wherein the operating member comprises a central axle, wherein the wall comprises a first section and a second section, the first section extending along a first plane perpendicular to the central axis, and the second section extending along a second plane offset from the first plane, and wherein the arcuate ledge and the first hook protrude from one side of the first section of the wall.

31. The endoscope of claim 30, wherein the second section of the operating member comprises teeth including ledges and flanges, the ledges and flanges guiding the proximal section of the first pull-wire.

32. A system comprising:
a display device; and
the endoscope of claim 29, the endoscope being connectable to said display device.

33. A method of assembling the endoscope of claim 29, the method comprising:
threading the proximal section of the first pull-wire onto the arcuate ledge, under the first hook, over the stem, and under the second hook, wherein under corresponds to a surface intermediate the central axis and a center of the first hook or the center of the second hook, and wherein over corresponds to a surface distal of a center of the stem;
tensioning the first pull-wire to a first predetermined tension;
tensioning the second pull-wire to a second predetermined tension;
joining the proximal section of the first pull-wire with the proximal section of the second pull-wire while the first pull wire is under the first predetermined tension and the second pull wire is under the second predetermined tension, the first predetermined tension and the second predetermined tension configured to set the bending section at a neutral position;
adjusting a position of the operating member with respect to the first pull-wire; and
affixing the first pull-wire to the operating member with the fastening member to prevent the proximal section of the first pull-wire from sliding along the arcuate ledge.

34. A method in assembling an endoscope, said method comprising:
providing a first handle housing part,
mounting an operating member in said first handle housing part,
providing a bending section,
connecting a first pull-wire and a second pull-wire to the bending section,
placing the first pull-wire in and partially surrounded by a first guide tube so as to present a first proximal pull-wire section extending from a proximal end said first guide tube
placing the second pull-wire in and partially surrounded by a second guide tube so as to present a second proximal pull-wire section extending from a proximal end of said first guide tube,
placing the first pull-wire section in engagement with at least one wire guide provided on said operating member,
tensioning the first pull-wire to a predetermined tension,
tensioning the second pull-wire to a predetermined tension,
while under tension joining the first proximal pull-wire section with the second proximal pull-wire section,
adjusting the position of the operating member with respect to the first pull-wire, and
securing the position of the first pull-wire with respect to the operating member.
